(19) United States
(12) Patent Application Publication
(10) Pub. No.: US 2016/0220723 A1
(43) Pub. Date: Aug. 4, 2016

(54) ABSORBENT POLYMER AND METHOD OF PREPARING THE SAME

(71) Applicants: SK GLOBAL CHEMICAL CO., LTD., Seoul (KR); SK INNOVATION CO., LTD, Seoul (KR)

(72) Inventors: Du-Youn KA, Daejeon (KR); Ju-Hee KIM, Daejeon (KR); Byoung-Tak YIM, Daejeon (KR); Ju-Eun JUNG, Daejeon (KR); Byoung-Cheon JO, Seoul (KR)

(21) Appl. No.: 15/011,726

(22) Filed: Feb. 1, 2016

(30) Foreign Application Priority Data
Jan. 30, 2015 (KR) 10-2015-0014929

(51) Int. Cl. A61L 15/60; A61L 15/26

(52) U.S. Cl. CPC A61L 15/60; A61L 15/26

(57) ABSTRACT

Disclosed are an absorbent polymer and a method of preparing the same. The absorbent polymer may be mixed with a cross-linking additive in order to allow the polymerized hydrogel to have a uniform cross-linkage structure, and thereby enhancing flow conductivity while having excellent absorption ability.

### CROSS-REFERENCE TO RELATED APPLICATION

This application claims priority to and the benefit of Korean Patent Application No. 10-2015-0014929, filed on Jan. 30, 2015, the disclosure of which is incorporated herein by reference in its entirety.

### BACKGROUND

1. Field of the Invention

The present invention relates to an absorbent polymer with improved permeability and a method of preparing the same.

2. Description of the Related Art

Super-absorbent polymer (‘SAP’) is an artificial polymer material capable of absorbing water of several tens to several hundreds of times the weight of the polymer. Also, the polymer material has high water-retention ability and can keep the absorbed water without releasing after absorbing the water, even though a pressure is somewhat applied thereto. Therefore, this polymer is broadly used in various products including hygienic products such as diapers, sanitary goods, etc.

With advanced performance of the hygienic products such as diapers as a major use of the super-absorbent polymer, excellent physical properties are required in various applications. More particularly, such physical properties may include free absorption, absorption rate, extractables, absorbency under pressure, absorbency under non-pressure, permeability (‘flow conductivity’), or the like. Among these physical properties, some are trade-off from each other. For example, as an internal cross-linkage degree of the absorbent polymer is reduced, the absorbency under non-pressure is increased whereas the absorbency under pressure is decreased. In order to improve all of the physical properties described above, a method of increasing a cross-linkage density of the surface layer of the polymer has been proposed.

In the production of the super-absorbent polymer, a polymer is formed by copolymerizing acrylic acid, an acrylic salt and a cross-linking agent wherein these compounds have different reactivities, respectively. In this case, a monomer with low reactivity is slowly consumed during polymerization and may remain in a form of oligomer before the end of the reaction. In addition, the absorbent polymer is exposed to a high temperature of 150° C. or more for 30 to 60 minutes in drying and surface-treatment processes during production thereof. In these processes, hydrolysis may occur and induce a cross-linkage structure of the polymer to be loosened. Such non-uniformity in reaction and thermal hydrolysis may cause non-uniformity in a network structure of the absorbent polymer, thus involving both of a dense part and a coarse part in the cross-linkage structure of the absorbent polymer prepared in any conventional process for production thereof.

The cross-linkage structure of the absorbent polymer is closely associated with absorption properties of the polymer. As the cross-linkage structure of the absorbent polymer becomes more compact, water absorbed in the polymer does not escape out of the polymer. On the other hand, when the cross-linkage structure is more loosen, the absorbed water is easily released to an outside. These characteristics are defined as absorption property under pressure, as the cross-linkage structure is non-uniform and includes increased amount of the coarse cross-linkage structure, absorption ability under pressure of the absorbent polymer may be reduced.

For analysis of the cross-linkage structure of the absorbent polymer, a method of absorbing solvent molecules having different sizes and calculating an absorption rate, a method of swelling an absorbent polymer and microtoming the same in a frozen state to observe a cross-section of the frozen polymer, or the like, have been proposed. However, such analysis methods are complicated and may observe only locally. Therefore, it is considered that these methods may not obviously explain a correlation between the cross-linkage structure and the physical properties of the absorbent polymer.

Other than excellent absorption ability, in order to achieve comfortable wearing sensation, the absorbent polymer recently tends to require high flow conductivity. The flow conductivity is an index to indicate how fast the water such as body fluid passes between the particles and, as this index shows more excellent characteristics, the water may rapidly pass through a contact surface consisting of absorbent polymers, and uniformly spread and be absorbed throughout an internal absorbent polymer. As a result, a user wearing the product provided with the absorbent polymer may feel the product wet not much.

### SUMMARY

Accordingly, it is an object of the present invention to provide an absorbent polymer with excellent absorbency under pressure and flow conductivity, and a method of preparing the same.

The above object of the present invention will be achieved by the following characteristics:

(1) A method of preparing an absorbent polymer, including: polymerizing a polymer composition which includes acrylic monomer and a cross-linking agent; adding a water-soluble compound containing at least two hydroxyl groups in an amount of 0.5 to 5 parts by weight relative to 100 parts by weight of un-neutralized acrylic acid, to a hydrogel obtained by the above polymerization, then, kneading the mixture; and drying and grinding the kneaded product.

(2) The method according to the above (1), wherein the acrylic monomer includes acrylic acid and acrylic salt.

(3) The method according to the above (2) wherein the acrylic salt is obtained by neutralizing the acrylic acid with a chemical base.

(4) The method according to the above (1), wherein the water-soluble compound containing at least two hydroxyl groups is selected from a group consisting of isosorbide, 2,3-butanediol, 1,4-butanediol and 1,3-propanol.

(5) The method according to the above (1), wherein the water-soluble compound containing at least two hydroxyl groups is included in a content of 2 to 5 parts by weight to 100 parts by weight of un-neutralized acrylic acid.

(6) An absorbent polymer having: an absorbency under pressure in a range of 20 to 45 g/g; a hydrolysis rate in a range of 0.01 to 0.2 cp/min; and a time to reach the maximum viscosity of hydrolysate in a range of 60 to 180 minutes.

(7) The absorbent polymer according to the above (6), wherein the absorbency under pressure ranges from 30 to 45 g/g.

(8) The absorbent polymer according to the above (7), further having an absorbency under non-pressure in a range of 30 to 50 g/g.

(9) The absorbent polymer according to the above (6), wherein the hydrolysis rate ranges from 0.01 to 0.15 cp/min.

(10) The absorbent polymer according to the above (6), wherein the hydrolysis rate ranges from 0.01 to 0.1 cp/min.

(11) The absorbent polymer according to the above (6), wherein the time to the maximum viscosity of hydrolysate ranges from 90 to 180 minutes.

(12) The absorbent polymer according to the above (6), further having a particle size in a range of 100 to 1000 μm.

The absorbent polymer prepared according to the inventive method of preparing the same, may improve uniformity in cross-linkage structure formed inside the polymer, so as to reduce extractables eluted from the polymer, accordingly, absorption ability under pressure and flow conductivity may be enhanced.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a view schematically illustrating an ideal absorbent polymer having a uniform cross-linkage structure formed therein;

FIG. 2 is a view schematically illustrating an absorbent polymer with highly non-uniform cross-linkage structure; and

FIG. 3 is a view schematically illustrating a configuration of an apparatus for measuring absorbency under pressure.

### DETAILED DESCRIPTION

The present invention discloses a method of preparing an absorbent polymer. The absorbent polymer prepared according to the inventive method has: an absorbency under pressure in a range of 20 to 45 g/g; a hydrolysis rate in a range of 0.01 to 0.2 cp/min; and a time to reach the maximum viscosity of hydrolysate in a range of 60 to 180 minutes.

Hereinafter, embodiments of an absorbent polymer according to the present invention will be described in detail.

According to one embodiment of the present invention, a hydrolysis rate refers to an increase in viscosity per minute over 60 minutes, and may be determined by introducing 10 g of absorbent polymer to a 1 kg hydrolysis solution, which was prepared by adding ultrapure water to 10 g of sodium hydroxide (NaOH), while rotating the hydrolysis solution at 25° C. and 500 rpm, stirring the solution for 30 minutes, taking 40 g of the reactant solution with a spoid having an inlet size of at least 5 mm while rotating the same, filtering the solution, measuring a viscosity of the solution by means of No. 18 spindle in a Brookfield viscometer equipped with a small sample adaptor accessory at 30° C. under a spindle velocity of 30 rpm, heating the reactant solution to 80° C. while continuously stirring, measuring the viscosity at an interval of 10 minutes over 60 minutes according to the same procedure as described above, and then, substituting the measured value for Equation 1 below to calculate the hydrolysis rate.

Hydrolysis rate=[Vis(60)−Vis (r.t)]/60 [Equation 1]

(wherein Vis (r.t) refers to a viscosity measured before heating the reactant to 80° C., and Vis (60) refers to a viscosity measured 60 minutes after heating the reactant to 80° C.)

A time to reach the maximum viscosity of hydrolysate in one embodiment of the present invention may be defined as follows: after heating the reactant to 80° C. under the same conditions for measurement of the hydrolysis rate as described above, then, measuring a viscosity at an interval of 10 minutes over 250 minutes, (1) when the viscosity with a variation in viscosity to the viscosity measured just before in ±1% or less is continuously measured twice or more, a time at the initial measurement point at which the variation in viscosity is in ±1% or less; otherwise, or (2) when the viscosity with a variation in viscosity to the viscosity measured just before in ±1% or less is not continuously measured twice or more, a time at the maximum value measurement point among viscosities measured at the interval of 10 minutes.

According to one embodiment of the present invention, the time to reach the maximum viscosity of the hydrolysate means a time to reach a condition that the hydrolysate (super-absorbent polymer) substantially has lost a cross-linkage structure.

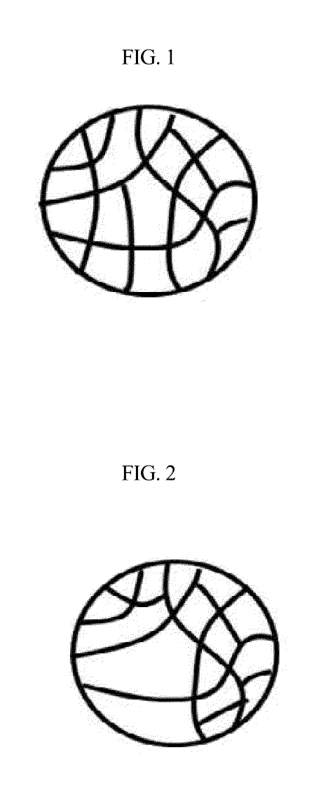

The inventors have found that absorbency under pressure at a specific section, a hydrolysis rate and a time to reach the maximum viscosity of hydrolysate have correlation to one another, and such correlation is closely associated with non-uniformity of the cross-linkage structure of the absorbent polymer and the flow conductivity of the absorbent polymer, thereby resulting in one embodiment of the present invention. FIG. 1 is a view schematically illustrating an ideal absorbent polymer having a cross-linkage structure uniformly formed therein. However, unlike that illustrated in FIG. 1, any conventional absorbent polymer prepared in the related art did not undergo an alternative process of improving uniformity of the cross-linkage structure, therefore, may have both of a dense network structural part having high degree of cross-linkage and a coarse network structural part having low degree of cross-linkage. Such an absorbent resin with high non-uniformity in cross-linkage structure is schematically illustrated in FIG. 2. The cross-linkage structure of the absorbent polymer is determined by a method of preparing the absorbent polymer, and the cross-linkage structure may have influence upon flow conductivity as well as absorption ability of the absorbent polymer.

The coarse cross-linkage structural part may play a role similar to extractables. When the absorbent polymer in swollen state receives a pressure, the absorbed water partially escapes out of the polymer. In this case, extractables remained in the polymer may be dissolved in the water and also released from the same. Such extractables released from the polymer may play an adhesive-like role between swollen absorbent polymer particles in order to prevent water from passing through the particles. Such a function refers to gel-blocking. In the absorbent resin, the coarse cross-linkage structural part is connected with the absorbent polymer. However, due to lack of cross-linking junctions, this part may easily fall apart or be excluded at length together with side branches, hence closing gaps between the swollen polymer particles similar to the extractables, hence causing gel-blocking phenomenon.

According to one embodiment of the present invention, the hydrolysis rate and the time to reach the maximum viscosity of hydrolysate may be a parameter to explain uniformity of the cross-linkage structure, which relates to a time for penetrating a hydrolysis solution into the polymer and a time for releasing the hydrolysate out of the polymer. If the cross-linkage structure of the absorbent polymer becomes loosen, or is not dense but includes a coarse structure, the hydrolysis rate becomes higher and the time to reach the maximum viscosity of the hydrolysate is decreased. On the other hand, if the cross-linkage structure is dense, or does not include a coarse structure but is uniform, the hydrolysis rate becomes lower and the time to reach the maximum viscosity of the hydrolysate is increased.

Therefore, one embodiment of the absorbent polymer according to the present invention may have a specific range of hydrolysis rate and a specific range of time to reach the maximum viscosity of hydrolysate, therefore, exhibit excellent uniformity in cross-linkage structure and flow conductivity.

The hydrolysis rate in one embodiment of the absorbent polymer according to the present invention may satisfy the range of 0.01 to 0.2 cp/min. If the hydrolysis rate is less than 0.01 cp/min, the cross-linkage structure is formed too much to actively proceed hydrolysis, hence considerably deteriorating absorption ability. If the hydrolysis rate exceeds 0.2 cp/min, non-uniformity of the cross-linkage structure may become more serious to cause a problem of deteriorating absorption property under pressure. The hydrolysis rate of the absorbent polymer, for example, ranges from 0.01 to 0.15 cp/min, and more particularly, from 0.01 to 0.1 cp/min.

One embodiment of the absorbent polymer according to the present invention may have a time to reach the maximum viscosity of hydrolysate (Tmax) in a range of 60 to 180 minutes. If the time to reach the maximum viscosity of hydrolysate is less than 60 minutes, the cross-linkage structure becomes loosen and non-uniform, hence causing a problem of deteriorating absorption ability under pressure. If the time exceeds 180 minutes, the cross-linkage structure is excessively dense and may cause a problem of deteriorating absorption property. The time to reach the maximum viscosity of hydrolysate, for example, ranges from 90 to 180 minutes.

One embodiment of the absorbent polymer according to the present invention may have absorbency under pressure ranging from 20 to 45 g/g, when measured in the experimental example.

When the absorbency under pressure of the absorbent polymer satisfies the values within the above range, a product such as a diaper containing the absorbent polymer may have a proper amount of water absorption so that a user of the product does not have unpleasant feeling, and may have a water-carrying ability enough to ensure the pressure particularly applied during daily-life activity.

In one embodiment of the absorbent polymer according to the present invention, if the absorbency under pressure is less than 20 g/g, the body fluid absorbed in the polymer may sometimes leak out due to the pressure applied during daily-life activity. On the other hand, if the absorbency under pressure exceeds 45 g/g, the polymer absorbs the water too much and decreases an intensity of swollen gel, hence causing such a problem that the polymer is readily cracked by impact applied during daily-life activity. Particularly, the absorbency under pressure of the one embodiment of the absorbent polymer according to the present invention may range from 30 to 45 g/g.

As necessary, one embodiment of the absorbent polymer according to the present invention may have absorbency under non-pressure in a range of 30 to 50 g/g, which is measured according to EDANA analysis method (WSP 241.2.R3). When the absorbency under non-pressure of the absorbent polymer satisfies the value within the above range, a product such as a diaper containing the absorbent polymer may have a proper amount of water absorption so that a user of the product does not have unpleasant feeling.

The absorbent polymer provided according to one embodiment of the present invention may be mixed with a cross-linking additive in order to allow the polymerized hydrogel to have a uniform cross-linkage structure, thereby satisfying physical properties described above. Hereinafter, one embodiment of a preparation method according to the present invention will be described in more details. Exemplary embodiments of the preparation method according to the present invention are proposed to more clearly understand technical spirit of the present invention together with the above detailed description, however, it is not construed that the present invention is particularly limited to contents described herein.

The absorbent polymer according to one embodiment of the present invention may be obtained by a manufacturing method including polymerization, kneading, drying and grinding processes. Such a manufacturing method may further include a surface cross-linking process.

The polymerization process may be conducted by polymerizing a polymer composition including acrylic monomer and a cross-linking agent.

The acrylic monomer may be selected from acrylic acid and salts thereof. This is advantageous in an aspect of excellent physical properties of a resin obtained by polymerizing the acrylic acid. Polymerization of acrylic acid may be facilitated by forming an acrylic salt through alkalization. For example, the acrylic salt may be obtained by neutralization of acrylic acid with alkali such as alkali-metal hydroxide, ammonia and organic amine. Among these, alkali-metal hydroxide, for example, sodium hydroxide, potassium hydroxide or lithium hydroxide is used in order to prepare an absorbent polymer having excellent physical properties while improving polymeric property of an acrylic monomer component. In order to improve the absorption ability of the absorbent polymer, alkalization may be conducted such that a neutralization rate of acid groups in the acrylic acid reaches 40 mol. % or more, and particularly, 60 mol. % or more.

The cross-linking agent used herein may include any one widely known in the related art, and be selected among compounds having functional groups possibly reacting with water-soluble substituent in the acrylic monomer. For example, the above cross-linking agent may be selected from a group consisting of bis-acrylamide having 6 to 12 carbon atoms, bis-methacrylamide, poly(meth)acrylate of polyol having 2 to 10 carbon atoms, and poly(meth)allylether of polyol having 2 to 10 carbon atoms, or the like, however, it is not particularly limited to the above listed compounds.

An amount of the cross-linking agent used herein is not particularly limited, but may range from 0.001 to 2 mol. %, and for example, 0.005 to 0.5 mol. % to a total acrylic monomer included and polymerized in the polymer. If a content of the cross-linking agent is less than 0.001 mol. % or exceeds 2 mol. %, it may be difficult to achieve sufficient absorption effects.

The polymer composition may have more appropriate physical properties for polymerization, when oxygen dissolved in a monomer ingredient under an inert gas atmosphere is substituted by such inert gas. This inert gas may be selected from, for example, nitrogen, carbon dioxide or argon gas.

Polymerization of the polymer composition may be performed by any one selected from thermal polymerization and photo-polymerization, or a combination of these two methods. More particularly, the thermal polymerization may be performed by selecting any one among typical heat polymerization to polymerize at a temperature of 40 to 90° C. for 2 to 30 minutes, or redox polymerization to polymerize at a relatively low temperature of 25 to 50° C. for 2 to 30 minutes. On the other hand, the photo-polymerization may be performed by irradiating UV-light at a temperature of 25 to 110° C. for 10 seconds to 20 minutes. When using the combination of the above both methods, a polymer composition including a photo-initiator and a thermal initiator mixed therein may undergo photo-polymerization by UV radiation to generate a neutralization heat, followed by thermal polymerization since the thermal initiator begins a reaction with the neutralization heat, thereby performing the polymerization. In order to produce a hydrogel phase polymer having low content of extractables and more excellent physical properties, the method using a combination of thermal initiator and photo-initiator is particularly selected.

The polymerization may be conducted by adding a polymerization initiator. The polymerization initiator added herein may be properly selected from conventional initiators used in the related art according to polymerization methods. The polymerization initiator used herein may include, for example, at least one initiator selected from a group consisting of azo-initiator, peroxide initiator, redox initiator, organic halide initiator, acetophenone, benzoin, benzophenone, benzyl compounds or derivatives thereof. A photo-polymerization initiator may include, for example, acetophenone, benzoin, benzophenone, benzyl compounds and derivatives thereof, in particular, at least one initiator selected from a group consisting of diethoxy acetophenone, 2-hydroxy-2-methyl-1-phenylpropanon, 4-(2-hydroxyethoxy)phenyl-(2-hydroxy)-2-propylketone, 4-benzoyl-4′-methyl-diphenyl sulfide, azo-compounds, or the like.

An amount of the polymerization initiator used herein is not particularly limited but, for example, may range from 0.001 to 2 mol. %, and for example, from 0.01 to 0.1 mol. % to a total monomer included and polymerized in the polymer. If the polymerization initiator is less than 0.001 mol. %, unreacted monomer residue may be increased. If the polymerization initiator exceeds 2 mol. %, polymerization may be difficult to control.

A hydrogel obtained by polymerizing the polymer composition may be mixed with a cross-linking additive during kneading.

As the cross-linking additive, a water-soluble compound may be adopted to be penetrated into the hydrogel and uniformly mixed together. If the additive is a non-water soluble material, the additive cannot be penetrated into and uniformly mixed with the hydrogel, instead, partially cross-linked on the surface of the hydrogel only. As a result, it may cause a deviation in physical properties in the finally produced particles.

More particularly, the cross-linking additive may be selected among water-soluble compounds having at least two hydroxyl groups. For example, the cross-linking additive used herein may be selected among compounds having at least two hydroxyl groups such as isosorbide, 2,3-butanediol, 1,4-butanediol, 1,3-propanediol, or the like, which are dissolved in water, however, it is not particularly limited to the above compounds.

The cross-linking additive added in the kneading process may undergo esterification with carboxylic acid in the residue, which functions as extractables in the hydrogel during production of the absorbent polymer, thus enabling the residue to be included in a network of the absorbent polymer, and thereby reducing elution of the extractables.

More particularly, the cross-linking additive may form additional cross-linkage junctions through esterification in a local part of the hydrogel, which lacks cross-linkage junctions by insufficiently using the cross-linking agent during polymerization or due to non-uniform polymerization.

Since the part of hydrogel with lack of the cross-linkage junctions has a coarse structure, the cross-linking agent may more easily penetrate into the above part than a dense cross-linkage structural part, therefore, to supplement the lack of cross-linkage junctions.

The cross-linking additive may be mixed in an amount of 0.5 to 5 wt. parts to 100 wt. parts of un-neutralized acrylic acid. When a content of the cross-linking additive is within the above range, the cross-linkage structure of the absorbent polymer may show relatively uniformity, to thus have a desired level of hydrolysis rate and hydrolysis time. As a result, it is possible to prepare an absorbent polymer having high flow conductivity without considerably reducing absorption ability under pressure. If the content of the cross-linking additive is less than 0.5 wt. part to 100 wt. parts of acrylic acid, cross-linking may not sufficiently proceed to increase an amount of extractables to be extracted. On the other hand, if the content of the cross-linking additive exceeds 5 wt. parts, too many cross-linkage junctions may be formed in the hydrogel and may deteriorate physical properties of the absorbent polymer. Particularly, the cross-linking additive may be mixed in an amount of 2 to 5 wt. parts to 100 wt. parts of un-neutralized acrylic acid.

The process of mixing the hydrogel and a cross-linking additive and kneading the same may be conducted using a kneading device such as a kneader, mincer, planetary mixer and hammer mixer, etc. and, selection and use of the kneading device are not particularly limited so long as this device can uniformly mix the hydrogel and the cross-linking additive.

The hydrogel after the kneading process may undergo a drying process to control a water-retention rate. During the drying, a drying temperature and a drying time may be selected under proper conditions on the base of the water-retention rate of the prepared hydrogel. For example, the drying process proceeds at a temperature of 160 to 190° C. for 20 to 60 minutes. If the drying temperature is less than 160° C., dry effects may be reduced to extend the drying time. If the drying temperature exceeds 190° C., the surface of the hydrogel is excessively dried to decrease absorbency under pressure of the absorbent polymer. The water-retention rate of the hydrogel obtained after the drying may range from 1 to 10% by weight.

The absorbent polymer may be generally ground and used in a form of powder. The dried hydrogel may be ground through a milling process, and such grinding may be conducted by any conventional milling method without particularly limitation in a technical configuration thereof so long as it may be used for grinding a resin. For example, the milling device such as a pin mill, hammer mill, screw mill, freezer miller, etc. may be used for grinding. In general, the absorbent polymer used for a product may have a particle size of 100 to 1,000 μm.

The ground absorbent polymer after the grinding may further undergo a surface cross-linking process to treat the polymer with a cross-linking agent, in order to regulate a cross-linking density. Such a cross-linking agent may be selected from a group consisting of diol having 2 to 8 carbon atoms or glycol compounds. For example, the diol compounds may include at least one selected from a group consisting of 1,3-propanediol, 2,3,4-trimethyl-1,3-pentanediol, 2-butene-1,4-diol, 1,4-butanediol, 1,3-butanediol, 1,5-pentanediol, 1,6-hexanediol, 1,2-cyclohexane dimethanol, and polycarbonate polyol. The glycol compound may include at least one selected from a group consisting of monoethylene glycol, diethyleneglycol, triethylene glycol, tetraethylene glycol, polyethyleneglycol, propylene glycol, dipropylene glycol, polypropylene glycol, glycerol and polyglycerol. Using the surface cross-linking agent described above, a surface cross-linkage density of the absorbent polymer may be regulated to thus enhance a particle strength and absorbency under pressure of the absorbent polymer.

Hereinafter, Exemplary embodiments are proposed to more concretely describe the present invention. However, the following examples are only given for illustrating the present invention and those skilled in the related art will obviously understand that various alterations and modifications are possible within the scope and spirit of the present invention. Such alterations and modifications are duly included in the appended claims.

### EXAMPLE

### Example 1

400 g of acrylic acid and 340 g of ultrapure water (Milli-Q integral 3; Millipore Co.) were mixed to prepare an acrylic acid solution. After dissolving 70 mol. % of sodium hydroxide (NaOH) to acrylic acid in 400 g of ultrapure water and cooling the same to 10° C., the mixture was slowly introduced into the acrylic acid solution. Nitrogen purging was conducted at 10° C. for 30 minutes, followed by adding 0.4915 g of potassium metabisulfite (K₂S₂O₈) and 0.2457 g of 1-hydroxycyclohexylphenylketone thereto. After adding 2.457 g of sodium hydrogen carbonate (NaHCO₃), UV light at 1 mw/cm² was rapidly irradiated for 1 minute. After removing the UV light, the mixture was left for 6 minutes to obtain a hydrogel. The obtained hydrogel was cut into pieces with a size of 1 cm³, and 0.5 wt. part of isosorbide to 100 wt. parts of acrylic acid was diluted with ultrapure water to prepare a water-soluble solution. By passing the cut hydrogel and 20 g of the water-soluble solution through a hood mixer (SFD(G); Shinsung Co.), the mixture was again passed through the hood mixer to completely knead the mixture. The resultant mixture was dried by a forced circulation drier (OF-02PW; Jeio Tech Co.). After increasing the temperature from an initial temperature of 30° C. up to 100° C. for 5 minutes and drying the same for 5 minutes, the temperature was again increased to 120° C. followed by drying for 5 minutes, increased 150° C. followed by drying for 5 minutes, and finally, increased 180° C. followed by drying for 20 minutes. In the chamber filled with the dried air, the sample was stored until the sample is cooled to room temperature. The cooled solid was ground and only particles having a size of 150 to 850 μm were selected using a mesh. Such grinding was conducted by a freezer/mill 6870 (SPEX SamplePrep Co.) under a liquid nitrogen atmosphere for 20 minutes. The selected particles were subjected to surface cross-linking using PCP-500 (Propylene carbonate polyol; SK Co.). After dissolving 4.23 g of surface cross-linking agent in 7 g of ethanol and gently adding 7 g of water thereto to prepare a surface cross-linking composition, the composition was uniformly mixed with the particles by a high-speed stirrer at a stirring intensity of “Low” (HMF-3260S; Hanil Co., Ltd.), followed by a reaction at 180° C. for 20 minutes, thereby preparing the absorbent polymer as a final product. Herein, the high-speed stirrer has a silicon blade rounded at its edge to prevent the absorbent polymer particles from being crushed by the blade.

### Comparative Example 1

The same procedures as described in Example 1 were conducted except that the cross-linking additive was not used.

### Examples 2 to 12 and Comparative Examples 2 to 5

The same procedures as described in Example 1 were conducted except that the cross-linking additive was used depending upon types and contents thereof listed in Table 1 below.

TABLE 1

| | Types of | Content of cross- |
|---|---|---|
| | cross- | linking additive |
| | linking | (wt. parts to 100 wt. |
| Section | additive | parts of acrylic acid) |
| Example 1 | Isosorbide | 0.5 |
| Example 2 | Isosorbide | 1 |
| Example 3 | Isosorbide | 2 |
| Example 4 | Isosorbide | 3 |
| Example 5 | Isosorbide | 5 |
| Example 6 | 2,3-butanediol | 0.5 |
| Example 7 | 2,3-butanediol | 1 |
| Example 8 | 2,3-butanediol | 2 |
| Example 9 | 2,3-butanediol | 3 |
| Example 10 | 2,3-butanediol | 5 |
| Example 11 | 1,3-propanol | 3 |
| Example 12 | 1,4-butanediol | 3 |
| Comparative | — | 0 |
| Example 1 | | |
| Comparative | Isosorbide | 0.1 |
| Example 2 | | |
| Comparative | Isosorbide | 10.0 |
| Example 3 | | |
| Comparative | 2,3-butanediol | 0.1 |
| Example 4 | | |
| Comparative | 2,3-butanediol | 10.0 |
| Example 5 | | |

### EXPERIMENTAL EXAMPLE

Physical properties of the absorbent polymer prepared in each of the examples and comparative examples have been measured by the following procedures, and results thereof are shown in Table 2 below.

1. Determination of Absorbency Under Pressure

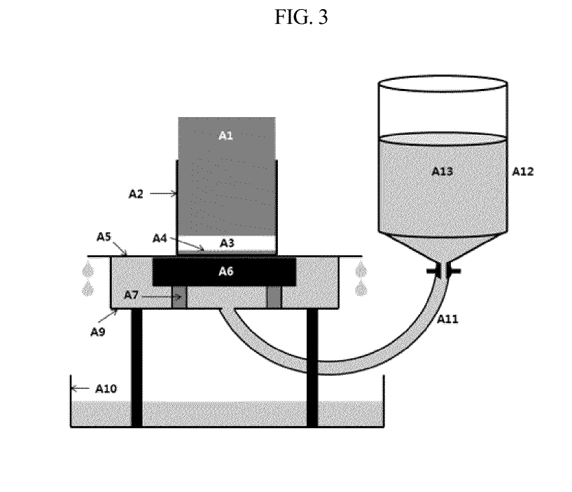

The absorbency under pressure was measured using the apparatus shown in FIG. 3. The measurement apparatus includes:

A1: weight (0.3 psi), A2: cylinder, A4: non-woven fabric, A5: paper filter, A6: glass filter, A7: glass filter support, A9: cylinder support, A10: container, A11: connection line, A12: reservoir. Installation of the measurement apparatus and measurement of the absorbency under pressure were conducted as follows.

The cylinder support A9 and the reservoir A12 were connected by the connection line A11, and each of the devices had a hole through which 0.9% saline A13 contained in the reservoir can pass and move. After placing the cylinder support A9 on the container A10, the top of the glass filter A6 was matched with the top of the cylinder support A9 in the same height using the glass filter support A7. Thereafter, the paper filter A5 having a larger size than the top of the cylinder support A9 was positioned on the same. By opening a plug of the reservoir A12 to flow the saline A13, the saline A13 passing through the connection line was fully filled in the top of the cylinder support A9 and the excess of saline was naturally discarded into an external container through the paper filter A5. Air bubbles were removed if these bubbles are formed between the glass filter A6 and the paper filter A5.

After evenly spreading 0.9 g (w0) on a non-woven fabric A3 above the cylinder A2 covered with non-woven fabric A4 at the bottom thereof, the cylinder was placed on the paper filter and a weight A1 was quickly provided thereon.

After 1 hour, the hydrogel in the cylinder was recovered, followed by measuring the weight of the hydrogel (w1, weight of the absorbent polymer after absorption). From the measured weight, the weight of a measuring sample (w0, weight of the absorbent polymer before absorption) was subtracted. The remainder was divided by the weight of the measuring sample (w0) to calculate the absorbency under pressure.

Absorbency under pressure (g/g)=(Weight of absorbent polymer after absorption (w1)−Weight of absorbent polymer before absorption (w0))/Weight of absorbent polymer before absorption (w0). [Equation 2]

2. Determination of Absorbency Under Non-Pressure (CRC) (EDANA WSP 241.2.R3)

Water-retention ability of the obtained absorbent polymer was determined according to EDANA analysis method (WSP 241.2.R3).

3. Determination of Hydrolysis Rate and Time to Reach Maximum Viscosity of Hydrolysate

The hydrolysis rate of the obtained absorbent polymer was determined by an increase in viscosity of the absorbent polymer to a reaction time of hydrolysate. More particularly, ultrapure was added to 10 g of sodium hydroxide (NaOH) to prepare 1 kg of hydrolysis solution, followed by rotating the solution at 25° C. and 500 rpm. Then, 10 g of the absorbent polymer prepared in each of the examples and comparative examples was quickly introduced between the center of vortex of the hydrolysis solution and a flask wall and agitated. After 30 minutes, 40 g of the reactant solution was taken with a spoid having an inlet size of at least 5 mm while rotating the same, filtered through a filter paper, followed by measurement of a viscosity (Vis r.t). The viscosity was measured by means of No. 18 spindle in a Brookfield viscometer equipped with a small sample adaptor accessory at 30° C. under a spindle velocity of 30 rpm. After heating the reactant solution to 80° C. while continuously stirring, the viscosity was measured at an interval of 10 minutes over 60 minutes according to the same procedure as described above (Vis 10 to 250), and then, the measured value was substituted for Equation 1 below to calculate the hydrolysis rate. However, if the maximum viscosity of hydrolysate was measured before 60 minutes (in the case of the comparative examples), the hydrolysis rate has been calculated as a variation in viscosity during the corresponding time.

Hydrolysis rate=[Vis (60)−Vis r.t]/60 [Calculation formula 1]

(wherein Vis (r.t) refers to a viscosity measured before heating the reactant to 80° C., while Vis (60) refers to a viscosity measured 60 minutes after heating the reactant to 80° C.)

4. Flow Conductivity

According to the measurement method described in U.S. Pat. Registration No. 8,466,228, the flow conductivity of the obtained absorbent polymer was measured.

TABLE 2

| | | | Hydro- | Time to | Flow |
|---|---|---|---|---|---|
| | Absorbency | Absorbency | lysis | reach | conduc- |
| | under | under non- | rate | maximum | tivity |
| | pressure | pressure | (cP/ | viscosity | (*10⁻⁸ |
| Section | (g/g) | (g/g) | min) | (min) | cm²) |
| Example 1 | 22.4 | 23.1 | 0.19 | 60 | 3.2 |
| Example 2 | 27.8 | 26.9 | 0.14 | 70 | 23.1 |
| Example 3 | 34.5 | 33.2 | 0.09 | 90 | 47.5 |
| Example 4 | 44.3 | 45.4 | 0.05 | 140 | 53.4 |
| Example 5 | 31.4 | 31.0 | 0.02 | 180 | 67.5 |
| Example 6 | 24.1 | 24.5 | 0.19 | 60 | 4.7 |
| Example 7 | 33.6 | 32.7 | 0.16 | 80 | 27.8 |
| Example 8 | 44.7 | 44.1 | 0.13 | 90 | 47.8 |
| Example 9 | 37.1 | 36.6 | 0.08 | 120 | 54.1 |
| Example 10 | 30.4 | 31.8 | 0.04 | 170 | 66.5 |
| Example 11 | 40.4 | 41.5 | 0.10 | 100 | 44.5 |
| Example 12 | 43.8 | 44.2 | 0.11 | 110 | 41.3 |
| Comparative | 13.2 | 14.1 | 0.5 | 30 | 0.1 |
| Example 1 | | | | | |
| Comparative | 16.4 | 16.8 | 0.45 | 40 | 0.3 |
| Example 2 | | | | | |
| Comparative | 10.3 | 11.1 | 0.005 | 220 | 94.2 |
| Example 3 | | | | | |
| Comparative | 16.3 | 15.2 | 0.37 | 40 | 0.3 |
| Example 4 | | | | | |
| Comparative | 11.6 | 10.4 | 0.007 | 200 | 91.1 |
| Example 5 | | | | | |

Referring to the above Table 2, it could be seen that the absorbent polymer prepared in each of Examples 1 to 12 according to the present invention satisfied the physical properties such as the absorbency under pressure of 20 to 45 g/g, the hydrolysis rate of 0.01 to 0.2 cp/min, and the time to reach the maximum viscosity of hydrolysate ranging from 60 to 180 minutes, and had excellent flow conductivity while maintaining superior absorbency under non-pressure.

Referring to Comparative Examples 1, 2 and 4, it could be found that, if the hydrolysis rate exceeds 0.2 cp/min and the time to reach the maximum viscosity of hydrolysate is less than 60 minutes, the flow conductivity was reduced.

Referring to Comparative Examples 3 and 5, if the hydrolysis rate is less than 0.01 cp/min and the time to reach the maximum viscosity of hydrolysate exceeds 180 minutes, the cross-linkage structure is too much dense, hence causing considerable deterioration in absorption ability of the absorbent polymer such as absorbency under pressure and absorbency under non-pressure.

The absorbent polymer prepared in each of Examples 1 to 12 according to the present invention has excellent absorption ability and flow conductivity, thereby being advantageously applied to hygienic products such as diapers.

What is claimed is:

1. A method of preparing an absorbent polymer, comprising:
polymerizing a polymer composition which includes acrylic monomer and a cross-linking agent;
adding a water-soluble compound containing at least two hydroxyl groups in an amount of 0.5 to 5 parts by weight relative to 100 parts by weight of un-neutralized acrylic acid, to a hydrogel obtained by the above polymerization, then, kneading the mixture; and
drying and grinding the kneaded product.

2. The method according to claim 1, wherein the acrylic monomer includes acrylic acid and acrylic salt.

3. The method according to claim 2, wherein the acrylic salt is obtained by neutralizing the acrylic acid with a chemical base.

4. The method according to claim 1, wherein the water-soluble compound containing at least two hydroxyl groups is selected from a group consisting of isosorbide, 2,3-butanediol, 1,4-butanediol and 1,3-propanol.

5. The method according to claim 1, wherein the water-soluble compound containing at least two hydroxyl groups is included in a content of 2 to 5 parts by weight to 100 parts by weight of un-neutralized acrylic acid.

6. An absorbent polymer having:
an absorbency under pressure in a range of 20 to 45 g/g;
a hydrolysis rate in a range of 0.01 to 0.2 cp/min; and
a time to reach the maximum viscosity of hydrolysate in a range of 60 to 180 minutes.

7. The absorbent polymer according to claim 6, wherein the absorbency under pressure ranges from 30 to 45 g/g.

8. The absorbent polymer according to claim 7, further having an absorbency under non-pressure in a range of 30 to 50 g/g.

9. The absorbent polymer according to claim 6, wherein the hydrolysis rate ranges from 0.01 to 0.15 cp/min.

10. The absorbent polymer according to claim 6, wherein the hydrolysis rate ranges from 0.01 to 0.1 cp/min.

11. The absorbent polymer according to claim 6, wherein the time to the maximum viscosity of hydrolysate ranges from 90 to 180 minutes.

12. The absorbent polymer according to claim 6, further having a particle size in a range of 100 to 1000 μm.
